(19) United States
(12) Patent Application Publication
(10) Pub. No.: US 2012/0157943 A1
(43) Pub. Date: Jun. 21, 2012

(54) Aspiration Path Resistive Element

(76) Inventors: Gary P. Sorensen, Laguna Niguel, CA (US); Susan Goodman Zurcher, Huntington Beach, CA (US)

(21) Appl. No.: 13/311,766

(22) Filed: Dec. 6, 2011

Related U.S. Application Data
(60) Provisional application No. 61/424,094, filed on Dec. 17, 2010.

(51) Int. Cl. A61F 9/007

(52) U.S. Cl. 604/319

(57) ABSTRACT

According to one exemplary aspect, this disclosure is directed to an aspiration system for a phacoemulsification surgical system. The system includes a pump and a flexible tubing configured to convey aspiration fluid from a hand piece to the pump. The flexible tubing includes a non-compliant, resistive element associated with the hand piece and disposed between the surgical site and the flexible tubing. The resistive element comprises a fluid pathway having substantially consistent nominal inner diameter and being configured to convey the aspiration fluid to the flexible tubing, the resistive element being formed in a compact orientation that provides a nonlinear fluid pathway length that is significantly greater than the axial length of the resistive element. The resistive element is structurally configured to provide occlusion surge resistance due to pressure changes resulting from occlusions in the aspiration path at the hand piece needle.

### PRIORITY CLAIM

This application claims the benefit of priority of U.S. Provisional Patent Application Ser. No. 61/424,094 titled “Aspiration Path Resistive Element”, filed on Dec. 17, 2010, whose inventors are Gary P. Sorensen and Susan Goodman Zurcher.

### BACKGROUND OF THE INVENTION

The present invention relates to phacoemulsification surgical systems and more particularly, to aspiration path resistive elements in phacoemulsification surgical systems.

Typical surgical instruments suitable for phacoemulsification procedures on cataractous lenses include an ultrasonically driven phacoemulsification hand piece with a cutting needle and an irrigation sleeve, and a control console. The hand piece is attached to the control console by an electric cable and flexible tubing. The flexible tubing supplies irrigation fluid to the surgical site and carries aspiration fluid from the surgical site to a waste or discard reservoir.

During a phacoemulsification procedure, the tip of the cutting needle and the end of the irrigation sleeve are inserted into the anterior segment of the eye through a small incision in the eye's outer tissue. The surgeon brings the tip of the cutting needle into contact with the lens of the eye, so that the vibrating tip fragments the lens. The resulting fragments are aspirated out of the eye through the interior bore of the cutting needle, along with irrigation fluid provided to the eye during the procedure.

Throughout the procedure, irrigating fluid is infused into the eye, passing between the irrigation sleeve and the cutting needle and exiting into the eye at the tip of the irrigation sleeve and/or from one or more ports or openings formed into the irrigation sleeve near its end. This irrigating fluid is critical, as it prevents the collapse of the eye during the removal of the emulsified lens, protects the eye tissue from the heat generated by the vibrating of the ultrasonic cutting needle, and suspends the fragments of the emulsified lens for aspiration from the eye.

During the surgical procedure, the console controls irrigation flow rates and aspiration flow rates to maintain a proper intra-ocular chamber balance in an effort to maintain a relatively consistent fluid pressure at the surgical site in the eye.

Aspiration flow rates of fluid from the eye are typically regulated by an aspiration pump that creates a vacuum in the aspiration line. The aspiration flow and/or vacuum are set to achieve the desired working effect for the lens removal. While a consistent fluid pressure in the eye is desirable during the phacoemulsification procedure, common occurrences or complications create fluctuations or abrupt changes in fluid flow and pressure at the eye. One known cause for these is occlusions or flow obstructions that block the needle tip. This common, and sometimes desirable occurrence, results in a sharp increase in vacuum in the aspirating line. When the occlusion is removed, the resulting high demand for fluid from the eye to relieve the vacuum can cause a sudden shallowing of the anterior chamber, as the aspiration flow momentarily sharply increases over the irrigation flow.

The degree of shallowing in the eye is a function of vacuum level within the aspiration path when the occlusion breaks, as well as resistive and compliance characteristics of the fluid path. Increased resistance in the aspiration path reduces the flow rate associated with occlusion break and thereby lessens the pressure drop from the irrigating source to the eye and the resulting shallowing of the anterior chamber.

The problem of occlusion surge has been addressed in the past in a number of ways including adding a reduced cross-section orifice. While such a reduced area reduces the effects of occlusion surge, reduction of aspiration path cross-section can also increase the potential for clogging during the procedure. Other methods have been used or proposed that involve torturous paths, with corners, angles, and fluid restrictors that are also subject to clogging. Some prior solutions involve a resistive element at or near the pump. However, the effectiveness of these solutions is limited due to the relatively large tubing compliance between the resistive element and the eye. Another attempted solution has been the use of increased lengths of flexible aspiration tubing in an attempt to increase overall tubing resistance. This solution of adding flexible tubing length has the undesirable effect of adding additional compliance to the aspiration path. The additional compliance increases the demand for fluid from the eye during occlusion break, sometimes entirely offsetting the benefits obtained by the longer tubing length.

### SUMMARY OF THE INVENTION

According to one exemplary aspect, this disclosure is directed to an aspiration system for a phacoemulsification surgical system. The system includes a pump configured to create a low pressure differential sufficient to draw aspiration fluid from a phacoemulsification surgical site. It also includes flexible tubing configured to convey the aspiration fluid from the hand piece to the pump. The flexible tubing is structurally configured to allow a user to manipulate the hand piece during a surgical procedure. It also includes a non-compliant, resistive element associated with the hand piece and disposed between the surgical site and the flexible tubing. The resistive element comprises a fluid pathway having substantially consistent nominal inner diameter and being configured to convey the aspiration fluid to the flexible tubing, the resistive element being formed in a compact orientation that provides a nonlinear fluid pathway length that is significantly greater than the axial length of the resistive element. The resistive element is structurally configured to provide occlusion surge resistance due to pressure changes resulting from occlusions in the aspiration path at the hand piece needle.

In some aspects, the resistive element is disposed within the hand piece. In other aspects, the resistive element is disposed outside but adjacent the hand piece. In some aspects, the resistive element is in one of a coil shape, a serpentine shape, and a spiral shape.

In some aspects, the resistive element has an inner diameter substantially matching the inner diameter of the flexible tubing, such that the aspiration line between the distal end of the resistive element and a cassette has a substantially uniform inner diameter.

In another exemplary aspect, the present disclosure is directed to a resistive element associated with a hand piece having a needle and disposed between a surgical site and flexible tubing in a phacoemulsification surgical system. The resistive element comprises a rigid body forming a nonlinear fluid pathway configured to convey aspiration fluid and emulsified tissue from a surgical site. The rigid body is formed of a material that remains substantially noncompliant when subjected to vacuum pressure applied by a pump of the phacoemulsification surgical system. The fluid pathway has a substantially consistent nominal inner diameter and is structurally configured to provide occlusion surge resistance due to pressure changes resulting from occlusions at the hand piece needle. The resistive element also includes an output port configured to connect with a flexible tube and arranged to pass the aspiration fluid and emulsified tissue through the output port and an input port configured to receive the aspiration fluid and emulsified tissue through the output port.

In another exemplary aspect, the present disclosure is directed to a method of reducing occlusion surge in a phacoemulsification surgical system. The method includes directing an aspiration fluid through an aspiration system of the phacoemulsification surgical system. It also includes aspirating fluid from the surgical site through a phacoemulsification needle and directing the aspirated fluid and emulsified tissue through a rigid, resistive element associated with the hand piece. The resistive element forms a fluid path having substantially consistent nominal inner diameter and is configured to convey the aspiration fluid to the flexible tubing. The resistive element is formed in a compact orientation that provides a nonlinear fluid pathway length that is significantly greater than the axial length of the resistive element and is structurally configured to provide occlusion surge resistance due to pressure changes resulting from occlusions in the aspiration path at the hand piece needle. The method also includes directing the aspiration fluid from the resistive element to flexible tubing.

It is to be understood that both the foregoing general description and the following detailed description are exemplary and explanatory only and are intended to provide further explanation of the invention as claimed. The following description, as well as the practice of the invention, sets forth and suggests additional advantages and purposes of the invention.

### BRIEF DESCRIPTION OF THE DRAWINGS

The accompanying drawings, which are incorporated in and constitute a part of this specification, illustrate several embodiments of the invention and together with the description serve to explain the principles of the invention.

FIG. 1 is an illustration of an exemplary phacoemulsification surgical console, according to one embodiment.

FIG. 2 is a block diagram of the phacoemulsification console of FIG. 1 showing various subsystems including a fluidics subsystem that drives aspiration according to the principles of the present disclosure.

FIG. 3 is a schematic of an exemplary fluidics subsystem usable with the phacoemulsification surgical console of FIGS. 1 and 2, according to an embodiment.

FIG. 4 is an illustration of an exemplary hand piece of the fluidics subsystem of FIG. 3, according to an embodiment.

FIGS. 5A-5C are illustrations of exemplary resistive element configurations usable in the fluidics systems disclosed herein.

FIG. 6 is a schematic of another exemplary fluidics subsystem usable with the phacoemulsification surgical console of FIGS. 1 and 2, according to an embodiment.

FIG. 7 is an illustration of an exemplary hand piece of the fluidics subsystem of FIG. 6, according to an embodiment.

### DETAILED DESCRIPTION OF THE PREFERRED EMBODIMENTS

Reference is now made in detail to several exemplary embodiments, examples of which are illustrated in the accompanying drawings. Wherever possible, the same reference numbers are used throughout the drawings to refer to the same or like parts.

The system of the present disclosure includes a resistive element in the aspiration path that helps reduce occlusion surge during a surgical procedure on the eye. The resistive element is associated with the hand piece and, in the exemplary embodiments described, includes a rigid, noncompliant, uniform cross-section aspiration fluid passageway. Because of its rigid nature, the fluid passageway maintains its shape when subjected to fluctuating vacuum pressures. This adds to the overall fluid passageway length of the aspiration system without adding to the overall occlusion surge. This additional passageway length of the aspiration system increases the overall tubing resistance, further reducing the potential occlusion surge. Substantial path length increases can be achieved in a relatively small amount of space via the use of coiled, spiraled, or serpentine shaped flow paths. Effectiveness of the added resistance is compounded by being close to the eye with minimal compliance between the resistive element and the eye. Flow passages in the system disclosed herein can have a larger inner cross-sectional area than would be required for a short, narrowing orifice to obtain the same level of resistance.

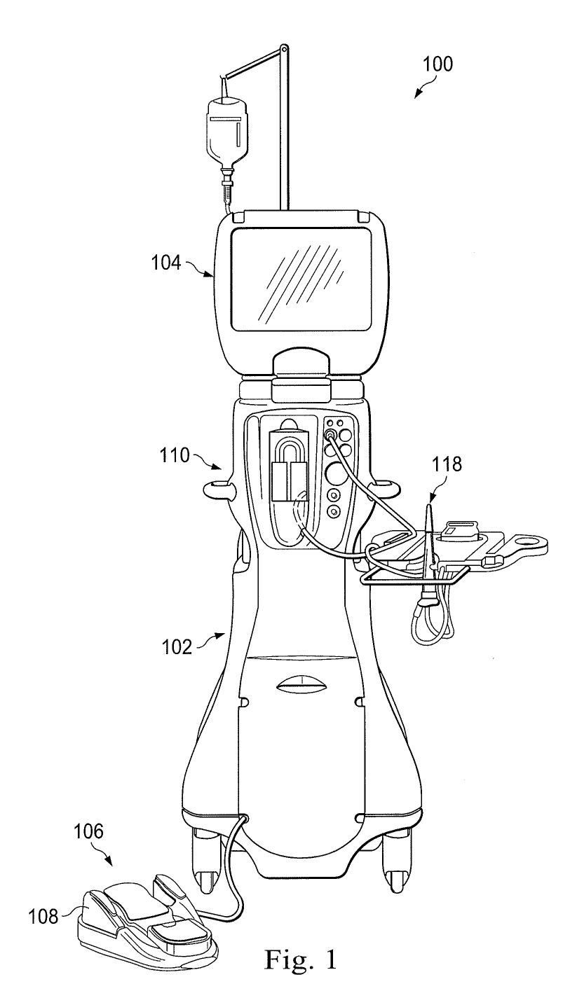

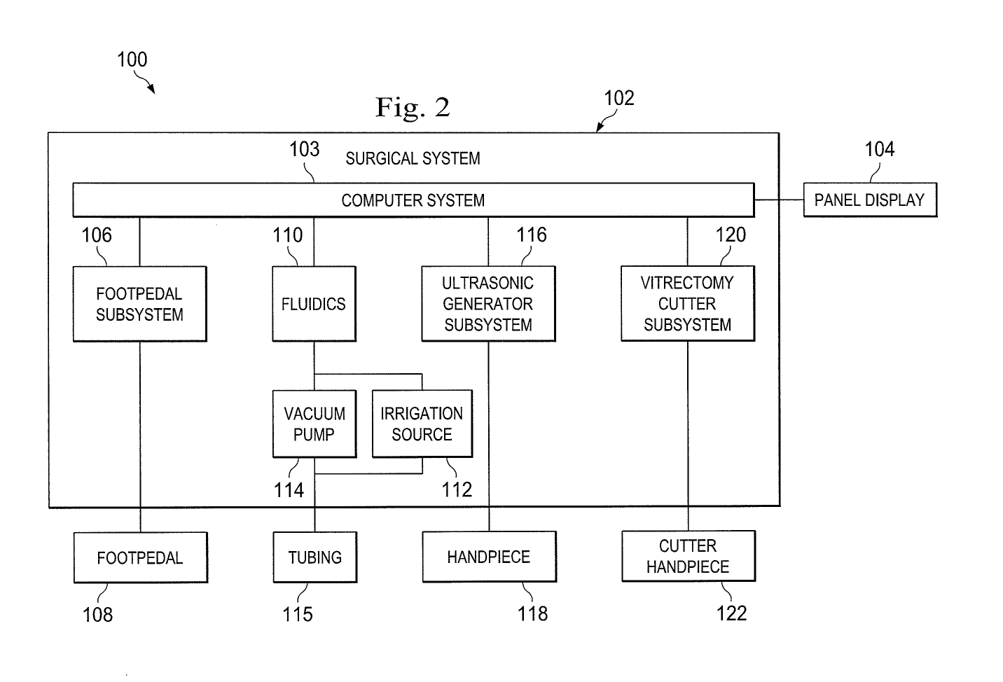

FIG. 1 illustrates an exemplary emulsification surgical console, generally designated 100. FIG. 2 is a block diagram of the console 100 showing various subsystems that operate to perform a phacoemulsification procedure. The console 100 includes a base housing 102 with a computer unit 103 and an associated display screen 104 showing data relating to system operation and performance during an emulsification surgical procedure. The console 100 also includes a number of subsystems that are used together to perform the emulsification surgical procedures. For example, the subsystems include a foot pedal subsystem 106 including, for example, a foot pedal 108, a fluidics subsystem 110 including an irrigation source 112 and a flow control vacuum pump 114 that irrigates and aspirates the eye through flexible tubing 115, an ultrasonic generator subsystem 116 including an ultrasonic oscillation hand piece 118 with a cutting needle, and a pneumatic vitrectomy cutter subsystem 120 including a vitrectomy hand piece 122. These subsystems overlap and cooperate to perform various aspects of the procedure. For example, in some embodiments, the end of the aspiration line of the flexible irrigation tubing is associated with the cutting needle of the hand piece to provide irrigation and cooling to the cutting and tissue during the procedure.

In addition, in some embodiments, an end of the flexible aspiration tubing is associated with the cutting needle of the hand piece 118 and aspirated through a hollow bore in the cutting needle.

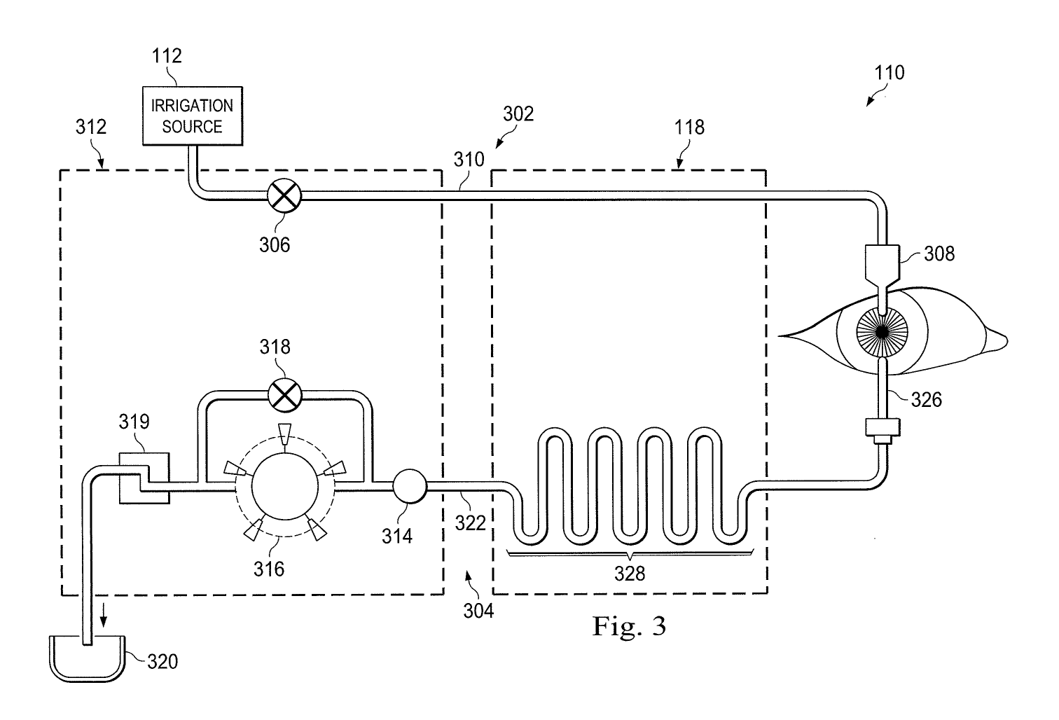

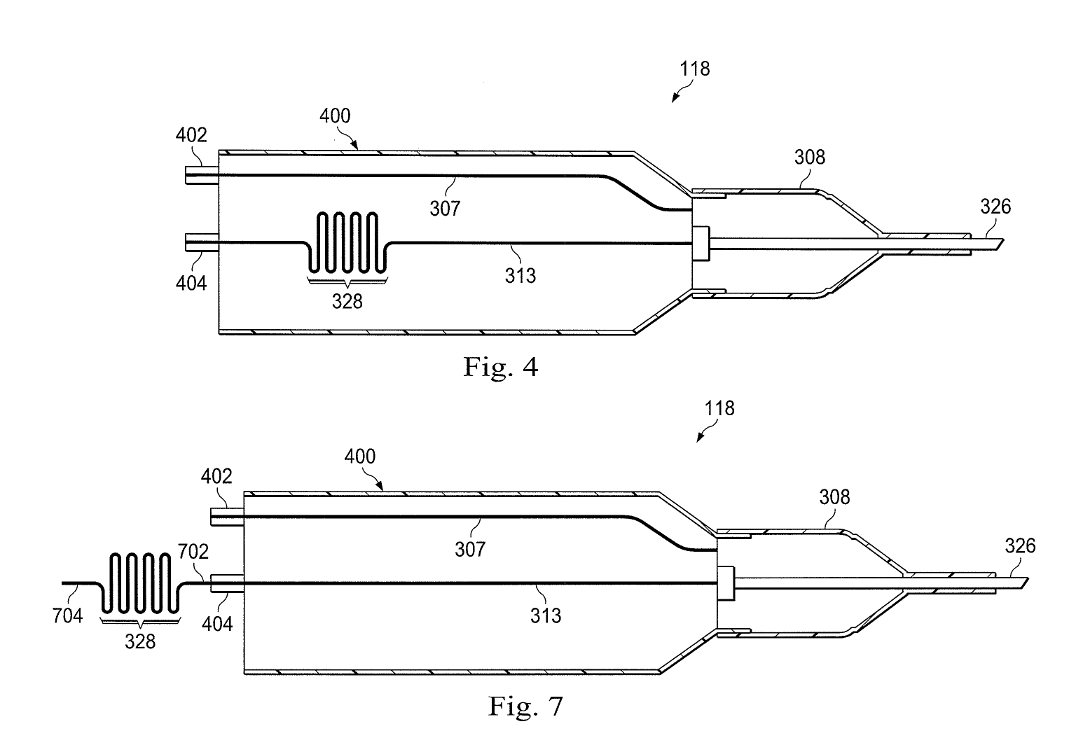

FIG. 3 illustrates a schematic showing the fluidics subsystem 110 and the hand piece 118. FIG. 4 shows the hand piece 118 in greater detail. The fluidics subsystem 110 includes an irrigation system 302 and an aspiration system 304, each in communication with the hand piece 118. The irrigation system 302 includes the irrigation source 112 as a sterile solution reservoir, an irrigation valve 306 that regulates flow from the reservoir to the surgical site, a fluid path 307 in the hand piece 118 (FIG. 4), and a sleeve 308 that may be considered a component of the hand piece 118 (best seen in FIG. 4).

The irrigation line 310 extends between the sterile solution reservoir 112 and the hand piece 118, which carries fluid to the surgical site (labeled in FIG. 3 as an eye). In one example, the sterile fluid is a saline fluid, however, other fluids may be used. At least a portion of the irrigation line 310 may be formed in part of the flexible tubing 115 in FIG. 2. In some embodiments, the line 310 is formed of multiple segments, with some segments being rigid and others being flexible. Also, in some embodiments, at least a portion of the irrigation line 310 is formed in a cassette 312 that cooperates with the console 100 in FIG. 1 to provide fluid communication between the sterile solution reservoir 112 and the hand piece 118 to the patient's eye. As indicated above, in some embodiments, the irrigation sleeve 308 is disposed about the cutting needle to provide irrigating fluid flow to the eye during the surgical procedure.

The aspiration system 304 includes a fluid path 313 (FIG. 4) in the handpiece 118, a pressure sensor 314, a pump 316, a vent valve 318, a drain line reservoir 319, and a drain reservoir 320. These are all connected by the aspiration line 322. As can be seen, the aspiration line 322 extends from the hand piece 118 to the drain reservoir 320. The aspiration line 322 carries away fluid used to flush the eye as well as any emulsified particles. As described above with reference to the irrigation line 310, at least a portion of the aspiration line 322 may be formed of the flexible tubing 115. In some embodiments, the aspiration line 322 is formed of multiple segments, with some segments being rigid and others being flexible. Also, in some embodiments, at least a portion of the aspiration line 322 is formed in the cassette 312 that cooperates with the console 100 in FIG. 1 to provide fluid communication between the hand piece 118 and the drain reservoir 320. It should be apparent that the drain reservoir 320 may in fact be a drain instead of a self-contained reservoir. As indicated above, in some embodiments, the aspiration line 322 including the aspiration fluid path 313, is in fluid communication with the bore of the cutting tip (labeled 326 in FIG. 3) of the hand piece 118 and is used to aspirate fluid and emulsified particles through the needle bore and into the aspiration line 322 during the surgical procedure.

In this embodiment as shown in FIGS. 3 and 4, the aspiration line 322 also includes a resistive element 328 that is structurally arranged to reduce the level of occlusion surge in a phacoemulsification procedure. As described above, during the surgical procedure, occlusions frequently and sometime intentionally block or limit aspiration flow into the tip 326 and into the aspiration line 322. During these moments when fluid and emulsified particles are not able to fully enter the aspiration system 304, the pump 316 continues to draw, increasing the vacuum in the aspiration line 322. As a result of the vacuum, the flexible tubing of the aspiration line 322 complies at least slightly, decreasing the volume within the flexible tube portion of the aspiration line 322. When the tip 326 is cleared, or upon the release of the occlusion, the built-up vacuum draws fluid from the surgical site in a single surge. This surge is compounded by the compliant nature of flexible tubing, which springs back to its standard volume under standard vacuum, drawing in additional fluid to compensate for the suddenly increased volume.

In this embodiment, the resistive element 328 reduces the level of occlusion surge in multiple ways. For example, the additional fluid pathway length due to the resistive element increases the overall tubing resistance, resulting in a dampened, or lower occlusion surge. Particularly, the aspiration line 322 between the tip 326 and the pump 316 provides a level of overall tubing resistance. As the length of the aspiration line 322 between the tip 326 and the pump 316 increases, so does the overall tubing resistance. Because the resistive element 328 adds additional length to the fluid pathway of the aspiration line 322, the overall tubing resistance is increased.

In addition, the resistive element 328 is formed of a rigid or noncompliant material. As such, as the vacuum increases, the resistive element does not deform. Deformation or compliance of the flexible tube can more than offset the benefits obtained by increasing the length of the fluid pathway. Accordingly, known systems that propose increasing the length of the flexible tube to increase tubing resistance may provide only limited benefits, and in some instances, because of the additional compliant tubing, may not provide any benefit in reducing occlusion surge. However, as disclosed in the embodiments herein, use of a rigid, noncompliant path length that increases the overall tubing resistance in the aspiration line 322 can provide the benefits of increasing the overall tubing resistance without the detriment arising from providing additional compliant tubing. As such, the rigid nature of the path length reduces or eliminates occlusion surge on the path length.

As shown in FIGS. 3 and 4, the rigid resistive element 328 is associated with the hand piece 118. As used herein, and as used in the claims, the term resistive element being “associated with the hand piece” means the resistive element is located relative to the hand piece close enough to provide occlusion surge benefits. The effectiveness of the added resistance is maximized by being in close proximity to the eye with minimal compliance (as may be introduced with flexible tubing) between the resistive element 328 and the eye. In some examples, benefits may be realized when the resistive element 328 and the surgical site are separated by less than twenty-four inches, and in some embodiments, less than about twelve inches of compliant tubing. In some embodiments, the resistive element 328 and the surgical site are separated by less than six inches of compliant tubing. In some embodiments, there is no compliant tubing separating the resistive element 328 and the surgical site. This maximizes the benefit obtained because the negative effects of the compliant tubing arise at locations relatively far down the fluid path and away from the eye. In FIGS. 3 and 4, the resistive element 328 is disposed in or is configured as a part of the hand piece 118.

In order to achieve the maximum benefit of the rigid resistive element while still making the hand piece convenient for the surgeon, the resistive element is configured to increase the length of the fluid pathway, without overly increasing the length of the hand piece, to avoid inconveniencing a surgeon using the hand piece. To accomplish this, the rigid resistive element is structurally arranged in a compact manner. In the example shown in FIGS. 3 and 4, the resistive element is formed in a serpentine shape. Other forms are contemplated, including a coil shape, a spiral shape, and a combination of these, among others. In some embodiments, the resistive element increases the noncompliant fluid pathway length by a distance within the range of about six inches to forty-eight inches. In some embodiments, the resistive element increases the noncompliant fluid pathway length by a distance within the range of about twelve inches to twenty-four inches.

FIGS. 5A-5C show some exemplary resistive elements in accordance with the principles of the present disclosure. FIG. 5A discloses a serpentine shaped resistive element 328a, FIG. 5B discloses a coil shaped resistive element 328b; and FIG. 5C discloses a spiral shaped resistive element 328c. As can be seen, each resistive element is formed in a compact orientation that provides a nonlinear fluid pathway length that is significantly greater than the axial length L of the resistive element. In some examples, the fluid pathway length is more than twice the axial length L, while in other examples, the fluid pathway length is more than four times the axial length. In yet other examples, the fluid pathway length is more than eight times the axial length of the resistive element. In the example of the spiral shaped resistive element, the spirals occur substantially in a plane, and from the center toward the outer edge or vice versa. The axial length is considered to be in the direction substantially normal to the plane. Although three examples of shapes are shown in FIGS. 5A-5C, others are contemplated as falling within the scope of this disclosure.

In the examples disclosed herein, the resistive element 328 includes a consistent, nominal inner diameter. Accordingly, the benefits of reducing occlusion surge can be obtained while limiting the chance of creating additional blockages in the resistive element and the aspiration line. Orifices, flow barriers, and mechanical restrictors of conventional systems can form dead zones with no or little flow and other areas of potential clogging. However, a consistent nominal diameter results in smooth fluid flow through the aspiration system, in some embodiments achieving laminar flow, providing smooth passage of tissue in the passageways. In the system disclosed herein, flow passages can have a larger inner cross-sectional area than would be required for a short, narrowing orifice to obtain the same level of resistance.

In some embodiments, the resistive element is formed of a rigid tube having a nominal diameter less than 0.100 inch. In some embodiments, the nominal diameter is within the range of 0.055-0.070 inch, with some aspects having a nominal diameter of about 0.062. In other embodiments, the nominal diameter is in the range of about 0.040-0.050 inch. Other dimensions, both larger and smaller, are contemplated.

In the embodiment disclosed in FIGS. 3 and 4, the nominal diameter of the resistive element is maintained as substantially equivalent to the nominal diameter of the flexible tubing between the hand piece 118 and the cassette 312. Accordingly, aspirated tissue particles have a reduced likelihood of becoming lodged within the aspiration path. As such, and for example only, one embodiment includes a resistive element 328 and a flexible aspiration tube extending to the cassette 312 that both have a nominal diameter in the range of about 0.040-0.050 inch. It should be noted that the flexible tube nominal diameter may be any diameter matching that of the resistive element 328, in these embodiments.

Referring now to FIG. 4, the hand piece comprises a housing 400 that supports the irrigation fluid path 307 and the aspiration fluid path 313. These extend from a distal end of the hand piece 118 having the irrigation sleeve 308 and the ultrasonic tip 326. The proximal end of the hand piece includes an irrigation connector 402 and an aspiration connector 404. In the embodiment shown, these connectors 402, 404 respectively connect to the flexible tubes of the irrigation line 310 and the aspiration line 322.

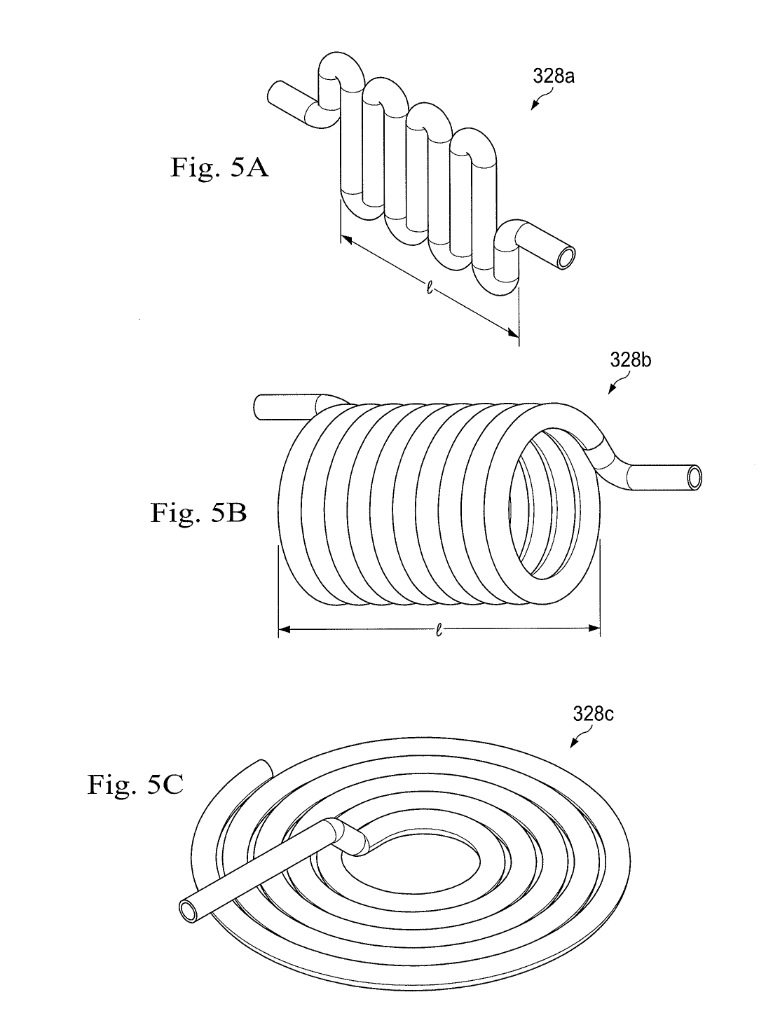

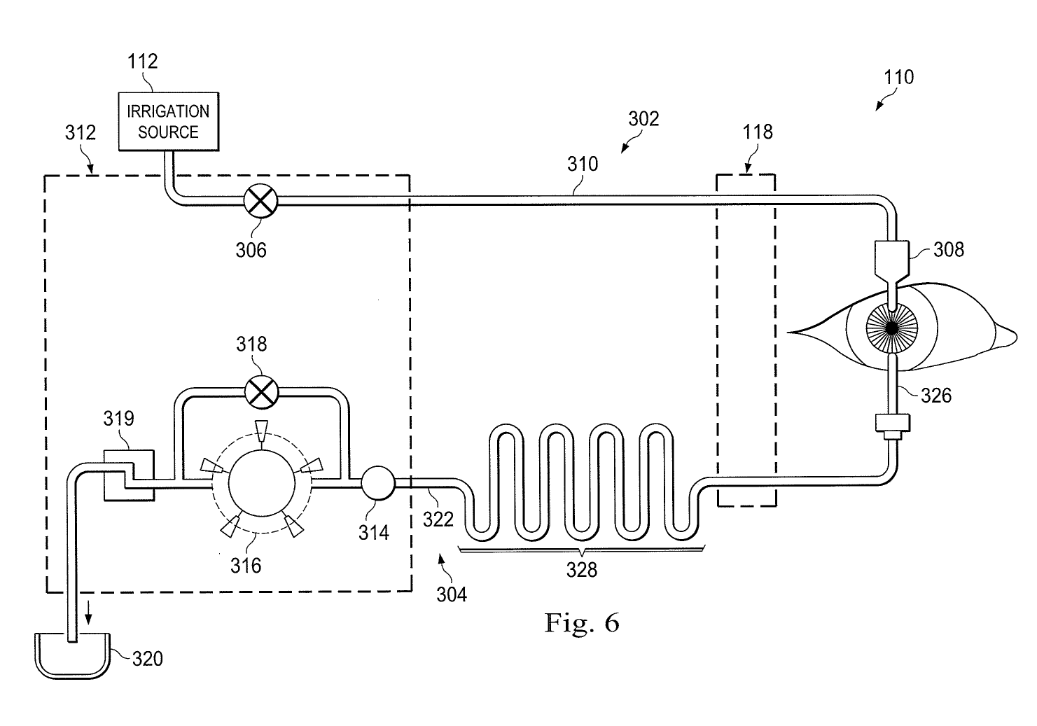

FIGS. 6 and 7 show another embodiment of the fluidics subsystem 110 and the hand piece 118. The embodiment is similar in many ways to the embodiment described above with reference to FIGS. 3 and 4. Accordingly, much of the discussion above applies equally to the embodiment of FIGS. 6 and 7, and will not be repeated here. However, the embodiment of FIGS. 5 and 6 differs from the embodiment in FIGS. 3 and 4 because the resistive element 328 is associated with the hand piece 118, but is disposed outside or adjacent the hand piece 118, instead of in or as a part of the hand piece 118 as described above. Here, in one embodiment, the resistive element 328 is a module that attaches between an end of the hand piece 118 and an end of the flexible tube of the aspiration line 322. Accordingly, in this embodiment, the resistive element 328 may be introduced into known, conventional systems by disposing the resistive element module in-line with the existing components of the conventional system.

Referring now to FIG. 7, the resistive element 328 includes a distal end 702 and a proximal end 704. The distal end 702 of the resistive element 328 fluidly connects to the aspiration connector 404 of the hand piece. The proximal end 704 of the resistive element 704 connects to the flexible tubing of the aspiration line 322. In some embodiments, the resistive element 128 module rigidly attaches directly to the hand piece 118. In other embodiments, the resistive element 128 attaches to a small segment of flexible tube between the resistive element 128 and the hand piece 118. In this embodiment, the flexible tube between the resistive element 128 and the hand piece 118 is selected so that the system still realizes the benefits of having the rigid resistive element 128 associated with the hand piece. For example, as discussed above, the small section of flexible tubing between the resistive element 328 and the aspiration connector 404 may be no longer than about twenty-four inches. In some embodiments, the section of flexible tubing may be less than twelve inches, or may be less than six inches. In some embodiments, the rigid resistive element directly connects to the aspiration connector directly.

The resistive element may be formed of any rigid noncompliant material, including, for example, metals and rigid polymer materials. In some examples, the resistive element is formed through an extrusion process, a molding process, or a machining process.

In use, the systems disclosed herein may operate to reduce occlusion surge during phacoemulsification procedures by employing the resistive element 328 to reduce the level of occlusion surge. It does this by linearly increasing the fluid pathway distance between the surgical site and all, or at least a large majority, of the compliant flexible tubing necessary for manipulation of the hand piece, while maintaining a nominal diameter through the resistive element. The system operates by directing fluids through the irrigation system 302 and the aspiration system 304 of the phacoemulsification surgical console 100. The irrigation system 302 directs fluid to the surgical site, and the aspiration system 304 removes fluid and tissue from the surgical site. During aspiration, fluid is directed through a needle of the hand piece 118 and into the hand piece. The fluid is then directed through the resistive element 328. The resistive element is associated with the hand piece in the manner described above to reduce the occlusion surge by reducing the effects of aspiration line compliance found in the aspiration system, by extending the length of the fluid passageway to increase the overall tube resistance, and by having a relatively consistent, nominal diameter to avoid clogging. For example, the resistive element is formed of a rigid, noncompliant material. The configuration of the resistive element lengthens the overall fluid pathway of the aspiration system, while only slightly or not adding additional length to the hand piece or the area adjacent to it.

In some instances, the resistive element is attached to one end of the flexible tube and to the end of the hand piece such as at the aspiration connector 404. In some embodiments, a relatively small length of flexible tube may connect the resistive element and the hand piece.

It should be appreciated that although several different embodiments are shown, any of the features of one embodiment may be used on any of the other embodiments shown. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

We claim:

1. An aspiration system for a phacoemulsification surgical system, comprising:
a pump configured to create a low pressure differential sufficient to draw aspiration fluid from a phacoemulsification surgical site;
flexible tubing configured to convey the aspiration fluid from the hand piece to the pump, the flexible tubing being structurally configured to allow a user to manipulate the hand piece during a surgical procedure; and
a non-compliant, resistive element associated with the hand piece and disposed between the surgical site and the flexible tubing, the resistive element comprising a fluid pathway having substantially consistent nominal inner diameter and being configured to convey the aspiration fluid to the flexible tubing, the resistive element being formed in a compact orientation that provides a nonlinear fluid pathway length that is significantly greater than the axial length of the resistive element, and the resistive element being structurally configured to provide occlusion surge resistance due to pressure changes resulting from occlusions in the aspiration path at the hand piece needle.

2. The aspiration system of claim 1, wherein the resistive element is disposed within the hand piece.

3. The aspiration system of claim 1, wherein the resistive element is disposed adjacent the hand piece.

4. The aspiration system of claim 1, wherein the resistive element is in one of a coil shape, a serpentine shape, and a spiral shape.

5. The aspiration system of claim 1, wherein the resistive element has an inner diameter substantially matching the inner diameter of the flexible tubing, such that the aspiration line between the distal end of the resistive element and a cassette has a substantially uniform inner diameter.

6. The aspiration system of claim 1, wherein the resistive element is disposed less than 12 inches of linear tube length from the hand piece.

7. The aspiration system of claim 6, wherein the resistive element is directly connected to the hand piece.

8. The aspiration system of claim 1, wherein the nominal inner diameter of the resistive element is within the range of 0.040-0.065 inch.

9. The aspiration system of claim 8, wherein the nominal inner diameter of the flexible tubing is within the range of 0.040-0.065 inch.

10. The aspiration system of claim 1, wherein the fluid pathway defined by the resistive element is greater than about six inches.

11. A resistive element associated with a hand piece having a needle and disposed between a surgical site and flexible tubing in a phacoemulsification surgical system, the resistive element comprising:
a rigid body forming a nonlinear fluid pathway configured to convey aspiration fluid and emulsified tissue from a surgical site, the rigid body being formed of a material that remains substantially noncompliant when subjected to vacuum pressure applied by a pump of the phacoemulsification surgical system, wherein the fluid pathway has a substantially consistent nominal inner diameter, the fluid pathway being structurally configured to provide occlusion surge resistance due to pressure changes resulting from occlusions at the hand piece needle;
an output port configured to connect with a flexible tube and arranged to pass the aspiration fluid and emulsified tissue through the output port; and
an input port configured to receive the aspiration fluid and emulsified tissue through the output port.

12. The resistive element of claim 11, wherein the rigid body is formed so that the pathway is in one of a coil shape, a serpentine shape, and a spiral shape.

13. The resistive element of claim 11, wherein the resistive element is formed in a compact orientation that provides a nonlinear fluid pathway length that is significantly greater than the axial length of the resistive element,

14. The resistive element of claim 11, wherein the nominal inner diameter of the resistive element is within the range of 0.040-0.065 inch.

15. The resistive element of claim 11, wherein the fluid pathway defined by the resistive element is greater than about six inches.

16. The resistive element of claim 11, wherein the fluid pathway defined by the resistive element is within the range of about six inches to about twenty-four inches.

17. The resistive element of claim 11, wherein the fluid pathway is devoid of dead spots.

18. A method of reducing occlusion surge in a phacoemulsification surgical system, the method comprising:
directing an aspiration fluid through an aspiration system of the phacoemulsification surgical system;
aspirating fluid from the surgical site through a phacoemulsification needle;
directing the aspirated fluid and emulsified tissue through a rigid, resistive element associated with the hand piece, the resistive element forming a fluid pathway having substantially consistent nominal inner diameter and being configured to convey the aspiration fluid to the flexible tubing, the resistive element being formed in a compact orientation that provides a nonlinear fluid pathway length that is significantly greater than the axial length of the resistive element, and being structurally configured to provide occlusion surge resistance due to pressure changes resulting from occlusions in the aspiration path at the hand piece needle; and
directing the aspiration fluid from the resistive element to flexible tubing.

19. The method of claim 18, further comprising:
attaching the flexible tubing to a port of the resistive element; and
attaching the hand piece to the resistive element.

20. The method of claim 18, wherein the resistive element is disposed in the hand piece.
